(19) United States
(12) Patent Application Publication
(10) Pub. No.: US 2017/0075219 A1
(43) Pub. Date: Mar. 16, 2017

(54) ONIUM SALT, PHOTOACID GENERATOR, PHOTOSENSITIVE RESIN COMPOSITION, AND METHOD FOR PRODUCING DEVICE

(71) Applicant: TOYO GOSEI CO., LTD., Ichikawa-shi, Chiba (JP)

(72) Inventor: Takashi Miyazawa, Inzai-shi, Chiba (JP)

(73) Assignee: TOYO GOSEI CO., LTD., Ichikawa-shi, Chiba (JP)

(21) Appl. No.: 15/310,733

(22) PCT Filed: May 13, 2015
(86) PCT No.: PCT/JP2015/063830

§ 371 Date: Nov. 11, 2016

Related U.S. Application Data
(60) Provisional application No. 61/992,702, filed on May 13, 2014.

(30) Foreign Application Priority Data
Nov. 20, 2014 (JP) 2014-235903

(51) Int. Cl. G03F 7/004; C07C 309/06; G03F 7/32; G03F 7/038; G03F 7/16; G03F 7/20; C07C 381/12; G03F 7/039

(52) U.S. Cl. CPC G03F 7/0045; C07C 381/12; C07C 309/06; G03F 7/0397; G03F 7/0382; G03F 7/162; G03F 7/168; G03F 7/2004; G03F 7/327

(57) ABSTRACT

An onium salt represented by formula (a). Z-A-W—Y⁺(R)ₙ X⁻ (a) (In formula (a), Z, A, W, Y, (R)ₙ, and X have the following meanings: Z represents a monovalent organic group having a ring structure provided with a conjugated π electron system, which may have one or more substituent groups; W represents a divalent organic group having a ring structure provided with a conjugated π electron system, which may have one or more substituent groups; A represents a divalent linking group containing a direct coupling of one or more bonds selected from a group consisting of a carbon-carbon single bond, a carbon-carbon double bond, and a carbon-carbon triple bond (any Z and/or W substituent group may form a ring structure in which one or more atoms included in Z and/or W are saturated or partially saturated together with A); Y is an iodine or sulfur atom, n=1 when Y is an iodine atom, and n=2 when Y is a sulfur atom; (R)ₙ may be identical to or different from one another, and each (R)ₙ is a monovalent organic group having a carbon number of 1 or more, which may have a substituent group; and X is a monovalent anion.)

### CROSS-REFERENCE TO RELATED APPLICATIONS

This application is a national phase entry under 35 U.S.C. §371 of International Patent Application PCT/JP2015/063830, filed May 13, 2015, designating the United States of America and published as International Patent Publication WO 2015/174471 A1 on Nov. 19, 2015, which claims the benefit under Article 8 and 35 U.S.C. §119(3) of the Patent Cooperation Treaty to Japanese Patent Application Serial No. 2014-235903, filed Nov. 20, 2014, and to U.S. Provisional Patent Application Ser. No. 61/992,702, filed May 13, 2014, the contents of which are incorporated herein by this reference.

### TECHNICAL FIELD

Some aspects of the application relate to chemistry and an onium salt. Some aspects of the application relate to a photoacid generator and a photosensitive resin composition containing an onium salt, and a method for producing a device using the same.

### BACKGROUND ART

In recent years, by utilizing a photolithography technique using a photoresist, production of a display such as a liquid crystal display (LCD) or an organic EL display (OLED), and formation of a semiconductor element have been performed actively. For a package or the like of the above electronic part or electronic product, light such as i-line having a wavelength of 365 nm or h-line (405 nm) or g-line (436 nm) having a longer wavelength than i-line has been widely used as an active energy ray. This is because a medium pressure, high pressure, or ultra-high pressure mercury lamp which is inexpensive as an irradiation light source and exhibits an excellent light emission intensity can be used. Therefore, it is considered that need for a photoacid generator exhibiting a high sensitivity to a wavelength region from an ultraviolet ray having a long wavelength to visible light will be further increased in the future.

Various photoacid generators are known as such a photoacid generator for a photoresist (refer to Patent Literature 1). The photoacid generator is a photosensitive agent for generating an acid by irradiation with light. On the other hand, in formation of a bump, with miniaturization of an electronic device, formation of a bump using a resist has attracted attention as an alternative method to conventional formation of a solder bump using a solder paste. In the resist for forming a bump, it has been desired to develop a photoacid generator for allowing light to reach a deepest part of a film even when the film is as thick as about 50 μm and generating an acid at a sufficient efficiency.

Patent Literature: Japanese Patent Publication Number 11-7124 publication

### Technical Problem

A sulfonate of a N-hydroxyphthalimide compound described in the above Patent Literature 1 absorbs light strongly in a wavelength range of 360 nm or more, and therefore light does not necessarily reach a deepest portion of a thick film. Due to a robust structure or the like, solubility is low, and it is not easy to prepare a composition for a photoresist.

A photoacid generator having a high sensitivity to ultraviolet light having a long wavelength such as i-line, having a high transmittance so as to be usable for a thick film, and capable of being produced at low cost is very limited.

inventionProvided is an onium salt having a high sensitivity to ultraviolet light having a long wavelength such as i-line in view of these circumstances. In addition, inventionprovided is an onium salt having a proper molar absorption coefficient for ultraviolet light having a long wavelength such as i-line and allowing light to reach a deepest part of a film as a photoresist photoacid generator for a thick film.

### Tech Solution

Intensive studies were made in order to solve the above problems. That is, one embodiment is an onium salt represented by the following formula (a).

Z-A-W—Y⁺(R)ₙX⁻ (a)

In the above formula (a), Z, A, W, Y, (R)ₙ, and X mean the following:

Z is a monovalent organic group having a cyclic structure provided with a conjugated π-electron system optionally containing at least one substituent;

W is a divalent organic group having a cyclic structure provided with a conjugated π-electron system optionally containing at least one substituent;

A is a direct bond or a divalent linking group containing at least one bond selected from the group consisting of a carbon-carbon single bond, a carbon-carbon double bond, and a carbon-carbon triple bond (any substituent of Z and W may form a cyclic structure saturated or partially saturated with A by using at least one atom contained in each of Z and W);

Y is an iodine atom or a sulfur atom, n=1 when Y is an iodine atom, and n=2 when Y is a sulfur atom;

(R)ₙ may be the same as or different from one another, and each of (R)ₙ is a monovalent organic group having one or more carbon atoms and optionally containing a sub stituent, typically an organic group such as a monovalent hydrocarbon group or an aryl group optionally containing a substituent; and

X is a monovalent anion.

One embodiment is an onium salt having a cation portion and an anion portion, in which the cation portion contains at least a first aromatic ring, a second aromatic ring, and a cation center, the cation center is bonded to either the first aromatic ring or the second aromatic ring, and a first carbon atom contained in the first aromatic ring is bonded to a second carbon atom contained in the second aromatic ring directly by a single bond or through a divalent linking group containing at least one bond selected from the group consisting of a carbon-carbon single bond, a carbon-carbon double bond, and a carbon-carbon triple bond.

One embodiment is a photoacid generator containing any one of the above onium salts.

One embodiment is a composition containing the above photoacid generator and a polymerizable synthetic compound reacted by an acid.

Preferably, the compound reacted by an acid contains a protective group deprotected by an acid or performs crosslinking by an acid.

One embodiment is a method for producing a device, including a first step for forming a coating film on a substrate using any one of the above compositions, a second step for exposing the coating film in a pattern shape using first light of an electromagnetic wave or a particle beam, and a pattern forming step for developing the coating film subjected to the second step to obtain a resist pattern. The coating film may be heated between the first and second steps and after the second step. A step other than the first to third steps may be added appropriately according to the compositions used or the like.

### Advantageous Effects

One embodiment can provide an onium salt having a high sensitivity to ultraviolet light having a long wavelength such as i-line. One embodiment can provide an onium salt allowing light to reach a deepest part of a film as a photoresist photoacid generator for a thick film.

### BRIEF DESCRIPTION OF DRAWINGS

FIG. 1 illustrates an absorption spectrum of an onium salt.

FIG. 2 illustrates a production process of a device of an integrated circuit (IC) using a photosensitive resin composition.

FIGS. 3A-3C are diagrams obtained by dividing FIG. 3. FIGS. 3A-3C illustrate a production process of a display device of an organic electroluminescent device (OLED) using a photosensitive resin composition.

FIGS. 3D to 3F are diagrams obtained by dividing FIG. 3. FIGS. 3D to 3F illustrate a production process of a display device of an organic electroluminescent device (OLED) using a photosensitive resin composition.

FIGS. 3G to 3I are diagrams obtained by dividing FIG. 3. FIGS. 3G to 3I illustrate a production process of a display device of an organic electroluminescent device (OLED) using a photosensitive resin composition.

### DESCRIPTION OF EMBODIMENTS

Hereinafter, embodiments will be described in detail.

<1> Onium Salt 1

An embodiment of the invention is an onium salt represented by the following formula (a).

Z-A-W—Y⁺(R)ₙX⁻ (a)

In the above formula (a), Z, A, W, Y, (R)n, and X mean the following:

Z is a monovalent organic group having a cyclic structure provided with a conjugated π-electron system optionally containing at least one substituent;

W is a divalent organic group having a cyclic structure provided with a conjugated π-electron system optionally containing at least one substituent;

A is a direct bond or a divalent linking group containing at least one bond selected from the group consisting of a carbon-carbon single bond, a carbon-carbon double bond, and a carbon-carbon triple bond (any substituent in Z and W may form a cyclic structure saturated or partially saturated with A by using at least one atom contained in each of Z and W);

Y is an iodine atom or a sulfur atom, n=1 when Y is an iodine atom, and n=2 when Y is a sulfur atom;

(R)ₙ may be the same as or different from one another, and each of (R)n is a monovalent organic group having one or more carbon atoms and optionally containing a sub stituent, typically an organic group such as a monovalent hydrocarbon group or an aryl group optionally containing a substituent; and

X is a monovalent anion.

In the above formula (a), Z is a monovalent organic group having a cyclic structure provided with a conjugated π-electron system optionally containing at least one substituent, typically, for example, a monovalent organic group containing a benzene aromatic ring optionally containing a substituent, a heteroaromatic ring optionally containing a substituent, or a non-benzene aromatic ring optionally containing a substituent.

Similarly, W is a divalent organic group having a cyclic structure provided with a conjugated π-electron system optionally containing at least one substituent, typically, for example, a divalent organic group containing a benzene aromatic ring optionally containing a substituent, a heteroaromatic ring optionally containing a substituent, or a non-benzene aromatic ring optionally containing a substituent.

The benzene aromatic ring is an aromatic ring in which a cyclic skeleton is formed by carbon atoms. Specific examples of the monovalent organic group containing the benzene aromatic ring include an aromatic organic group to which at least one benzene ring is linked directly, such as a phenyl group, a biphenyl group, a terphenyl group, or a quaterphenyl group. Among these groups, when i-line, h-line, or g-line is used as excitation light of the onium salt, a phenyl group, a biphenyl group, or a terphenyl group is preferable.

Furthermore, specific examples of the monovalent organic group containing the benzene aromatic ring include a condensed polycyclic aromatic ring in which at least two carbon atoms constituting one aromatic ring are also contained in carbon atoms constituting at least one adjacent aromatic ring, such as a naphthyl group, an anthryl group, a phenanthrenyl group, a pentalenyl group, an indenyl group, an indacenyl group, an acenaphthyl group, a fluorenyl group, a heptalenyl group, a naphthacenyl group, a pyrenyl group, a chrysenyl group, or a tetracenyl group. Among these groups, when i-line, h-line, or g-line is used as excitation light of the onium salt, a naphthyl group, an anthryl group, or a phenanthrenyl group is preferable.

The heterocyclic aromatic ring contains an atom other than a carbon atom, for example, at least one hetero atom such as an oxygen atom, a sulfur atom, or a nitrogen atom in a cyclic skeleton thereof. Specific examples of the monovalent organic group containing the heteroaromatic ring include a monocyclic and a condensed heteroaromatic group, such as a furanyl group, a thienyl group, a pyranyl group, a thiopyranyl group, a pyrrolyl group, an imidazolyl group, an oxazolyl group, a thiazolyl group, a pyrazolyl group, a pyridyl group, an isobenzofuranyl group, a benzofuranyl group, an isochromenyl group, a chromenyl group, an indolyl group, an isoindolyl group, a benzimidazolyl group, a xanthenyl group, an acridinyl group, or a carbazolyl group. When i-line, h-line, or g-line is used as excitation light of the onium salt, a furanyl group, a thienyl group, a pyranyl group, a thiopyranyl group, a pyrrolyl group, an imidazolyl group, an oxazolyl group, a thiazolyl group, a pyrazoyl group, a pyridyl group, an isobenzofuranyl group, a benzofuranyl group, or the like is preferable.

The non-benzene aromatic ring contains a cyclic conjugated system other than a benzene nucleus. Specific examples of the non-benzene aromatic ring include azulene, annulene, a tropylium cation, and metallocene.

The organic groups exemplified above can be introduced into the onium salt as a monovalent organic group and a divalent organic group for Z and W, respectively.

In the monovalent organic group or the divalent organic group, at least one hydrogen atom may be replaced with various substituents. Specific examples of the substituents include a cyano group, a trifluoromethyl group, a nitro group, an acetyl group, an iodo group, a bromo group, a chloro group, a fluoro group, an amide group, an alkyl group, an aryl group, an alkoxy group, a hydroxy group, a thiol group, an alkylthio group, an amino group, an alkylamino group, and a dialkylamino group.

Among the substituents, an electron-donating group such as an alkyl group (—R1), an aryl group (—Ar1), an alkoxy group (—OR1), an aryloxy group (—OAr1), a hydroxy group, a thiol group, an alkylthio group (—SR1), an amino group, an alkylamino group (—NHR1), a dialkylamino group (—NR1R2), an arylamino group (—NHAR1), a diarylamino group (—NAr1Ar2), or an N-alkyl-N-arylamino group (—NR1Ar1) is preferably bonded directly to the π-electron system of Z or W because an electron is donated to a cation center of the onium salt and an acid generation efficiency is improved, for example. The electron-donating group is more preferably bonded directly to the π-electron system of Z.

As used herein, the “cation center” means “Y” in the above formula (a).

It is preferably any one selected from the group consisting of R1 and R2 (in the formula, R1 and R2 each independently represent an alkyl group having one or more carbon atoms and optionally containing a substituent) contained in the electron-donating group.

Specific examples of the alkyl group having one or more carbon atoms, and optionally containing a substituent as R1 and R2 include a straight chain alkyl group such as a methyl group, an ethyl group, an n-propyl group, an n-butyl group, an n-pentyl group, an n-hexyl group, an n octyl group, or an n-decyl group; a branched alkyl group such as an isopropyl group, an isobutyl group, a tert-butyl group, an isopentyl group, a tert-pentyl group, or a 2-ethylhexyl group; a silyl group-substituted alkyl group obtained by replacing one of hydrogen atoms thereof with a trialkylsilyl group such as a trimethylsilyl group, a triethylsilyl group, or a dimethylethylsilyl group; and an alkyl group obtained by replacing at least one of hydrogen atoms thereof with a cyano group or a halogen group.

It is preferably any one selected from the group consisting of Ar1 and Ar2 (in the formula, Ar1 and Ar2 each independently represent an aryl group optionally containing a substituent) contained in the electron-donating group.

Specific examples of the aryl group optionally containing a substituent as AR1 and Ar2 include a phenyl group, a biphenyl group, a terphenyl group, a quaterphenyl group, a naphthyl group, an anthryl group, a phenanthrenyl group, a pentalenyl group, an indenyl group, an indacenyl group, an acenaphthyl group, a fluorenyl group, a heptalenyl group, a naphthacenyl group, a pyrenyl group, a chrysenyl group, a tetracenyl group, a furanyl group, a thienyl group, a pyranyl group, a thiopyranyl group, a pyrrolyl group, an imidazolyl group, an oxazolyl group, a thiazolyl group, pyrazoyl group, a pyridyl group, an isobenzofuranyl group, a benzofuranyl group, an isochromenyl group, a chromenyl group, an indolyl group, an isoindolyl group, a benzimidazolyl group, a xanthenyl group, an acridinyl group, and a carbazolyl group.

Among the electron-donating groups, an alkoxy group, an aryloxy group, and an alkylthio group are particularly preferable because of relatively high stability to an acid generated by the onium salt or the like. An alkoxy group, an aryloxy group, and the like are more preferable because a carbon atom-oxygen atom bond is more stable than a carbon atom-sulfur atom bond from a viewpoint of stability of the onium salt.

A is a direct bond or a divalent linking group containing at least one bond selected from the group consisting of a carbon-carbon single bond, a carbon-carbon double bond, and a carbon-carbon triple bond. The “divalent linking group containing at least one carbon-carbon double bond or carbon-carbon triple bond” is not particularly limited as long as being a divalent linking group containing at least one of -C=C—and —C≡C—, and examples thereof include —C═C—, —C═C—C═C—, —C≡C—, —C≡C—C≡C—, and —C═C—C≡C—. A is preferably a direct bond or an ethynylene group (—C≡C—).

Any substituent in Z and W may form a cyclic structure saturated or partially saturated with A by using at least one atom contained in each of Z and W. For example, when each of Z and W is a phenyl group which is a cyclic organic group or a phenylene group which is a cyclic organic group, the following structures (1) and (2) are exemplified (in the following general formula, “—Y+(R)nX—” is omitted). In the following structures, each of Z and W is a phenyl group or a phenylene group, but the onium salt according to the embodiment of the invention is not limited thereto. The cyclic structures are not limited to the following four-membered ring and five-membered ring, but may contain at least one hetero atom such as an oxygen atom, a nitrogen atom, a silicon atom, or a sulfur atom in a skeleton of the cyclic structure.

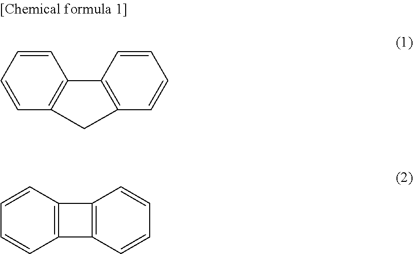

As Z-A-W-(in the general formula, “—Y⁺(R)ₙX⁻” is omitted) of the onium salt according to the embodiment of the invention, the following structures (3) to (11) are preferably exemplified. Each of the following structures (3) to (11) may be the linking group containing at least one bond selected from the group consisting of a carbon-carbon single bond, a carbon-carbon double bond, and a carbon-carbon triple bond in place of a direct single bond. In the following structures, Z may be replaced with W through a direct single bond. That is, —Y+(R)nX— in the above formula (a) is only required to be bonded to any cyclic organic group in the following structures.

At least one hydrogen atom of the following linked cyclic organic groups may be replaced with a substituent exemplified above. In the following formula, a carbon atom constituting at least one cyclic structure of the linked cyclic organic groups may be replaced with at least one hetero atom selected from an oxygen atom, a sulfur atom, and a nitrogen atom.

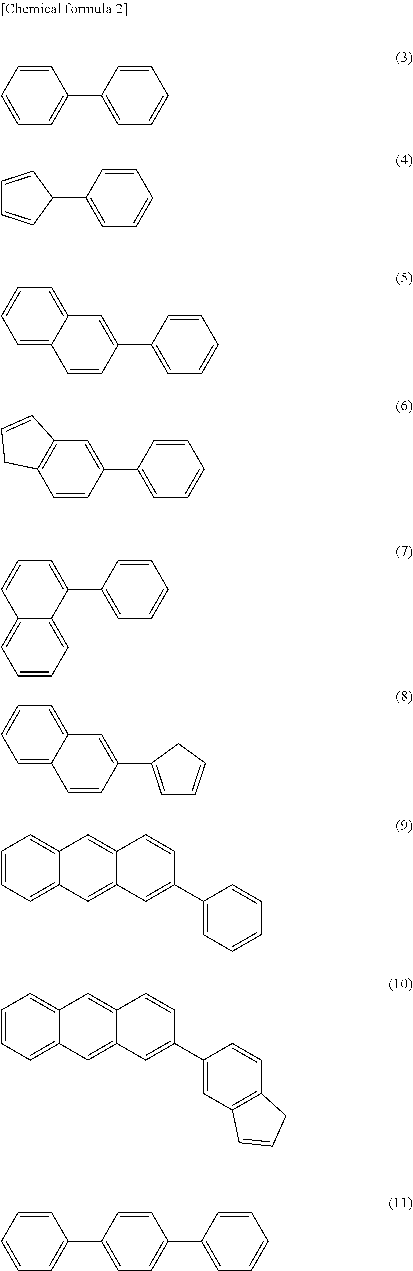

In the onium salt according to one embodiment, preferably, Z is a phenyl group and/or W is a phenylene group.

In the onium salt according to one embodiment, Y is preferably a sulfonium cation as a sulfur atom or an iodonium cation as an iodine atom.

In the onium salt according to one embodiment, X⁻ is preferably an anion selected from the group consisting of CF₃CO₂⁻, CH₃CO₂⁻, CF₃CF₂C₄H₄SO₃, CH₃SO₃⁻, (C₆F₅)₄B⁻, SbF₆⁻, PF₆⁻, BF₄⁻, CF₃SO₃⁻, HSO₄⁻, (CF₃CF₂)₃PF₃⁻, (CF₃CF₂)₂PF₄⁻, (CF₃CF₂)PF₅⁻, ((CF₃)₂C₆H₃)₄B⁻, (C₆F₅)₄Ga⁻, ((CF₃)₂C₆H₃)₄Ga⁻, a nonafluorobutanesulfonic acid anion, a butanesulfonic acid anion, a camphorsulfonic acid anion, a benzenesulfonic acid anion, a p-toluene sulfonic acid anion, (CF₃SO₂)₃C⁻, (CF₃SO₂)₂N⁻, and (C₄F₉SO₂)₂N⁻

(R)ₙ which are substituents on Y may be the same as or different from one another, and each of (R)n is a monovalent organic group having one or more carbon atoms and optionally containing a substituent, typically an organic group such as a monovalent hydrocarbon group or an aryl group optionally containing a substituent.

Specific examples of each of (R)ₙ include a straight chain alkyl group such as a methyl group, an ethyl group, an n-propyl group, an n-butyl group, an n-pentyl group, an n-hexyl group, an n-octyl group, or an n-decyl group; a branched alkyl group such as an isopropyl group, an isobutyl group, a tert-butyl group, an isopentyl group, a tert-pentyl group, or a 2-ethylhexyl group; a silyl group-substituted alkyl group obtained by replacing one of hydrogen atoms of the alkyl groups with a trialkylsilyl group such as a trimethylsilyl group, a triethylsilyl group, or a dimethylethylsilyl group; an alkyl group obtained by replacing at least one of hydrogen atoms of the alkyl groups with a cyano group or a halogen group; and an aromatic ring group such as a phenyl group, a biphenyl group, a terphenyl group, a quaterphenyl group, a naphthyl group, an anthryl group, a phenanthrenyl group, a pentalenyl group, an indenyl group, an indacenyl group, an acenaphthyl group, a fluorenyl group, a heptalenyl group, a naphthacenyl group, a pyrenyl group, a chrysenyl group, a tetracenyl group, a furanyl group, a thienyl group, a pyranyl group, a thiopyranyl group, a pyrrolyl group, an imidazolyl group, an oxazolyl group, a thiazolyl group, a pyrazoyl group, a pyridyl group, an isobenzofuranyl group, a benzofuranyl group, an isochromenyl group, a chromenyl group, an indolyl group, an isoindolyl group, a benzimidazoyl group, a xanthenyl group, an acridinyl group, or a carbazolyl group. Each of (R)n may be an alkyl group containing an aromatic ring, such as a benzyl group or a phenethyl group.

R may contain an electron-withdrawing group such as a cyano group, a nitro group, a perfluoroalkyl group, or a halogen atom and such an electron-donating group described above as a substituent.

When Y is a sulfur atom, such a 4-membered to 8-membered ring cyclic structure containing a sulfur atom as illustrated in the following (12) to (16), in which each of (R)n is bonded to one another, may be possible.

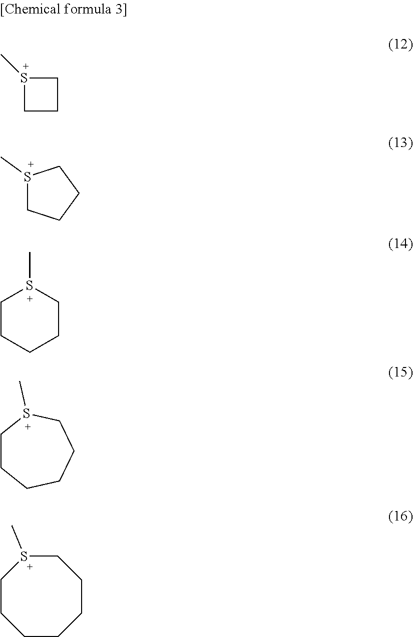

Y is preferably bonded to an aromatic ring group such as a phenyl group exemplified above as R from a viewpoint of improving long-term stability of the onium salt. Particularly when Y is a sulfur atom, all of Rs are preferably aromatic ring groups.

In the onium salt according to one embodiment, specific preferable examples of the onium salt represented by the above formula (a) will be illustrated below.

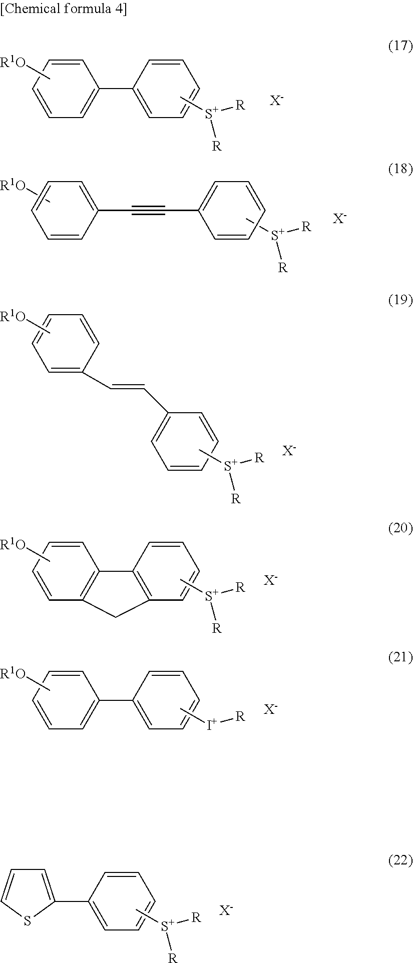

<2> Onium Salt 2

An onium salt according to an embodiment has a cation portion and an anion portion. The cation portion contains at least a first aromatic ring, a second aromatic ring, and a cation center. The cation center is bonded to either the first aromatic ring or the second aromatic ring. A first carbon atom contained in the first aromatic ring is bonded to a second carbon atom contained in the second aromatic ring directly by a single bond or through a divalent linking group containing at least one bond selected from the group consisting of a carbon-carbon single bond, a carbon-carbon double bond, and a carbon-carbon triple bond. Typically, the cation portion contains a first organic group provided with a cyclic structure containing the first aromatic ring optionally containing at least one substituent and a second organic group provided with a cyclic structure containing the second aromatic ring optionally containing at least one substituent. The cation center is preferably a sulfur atom, an iodine atom, or the like. Examples of the first organic group include an organic group containing a benzene aromatic ring forming a cyclic skeleton thereof only by carbon atoms, a heteroaromatic ring containing an atom other than a carbon atom in a cyclic skeleton thereof, or a non-benzene aromatic ring containing a cyclic conjugated system other than a benzene nucleus. Similarly, examples of the second organic group include an organic group containing a benzene aromatic ring, a heteroaromatic ring, or a non-benzene aromatic ring. Specific examples of the organic group containing a benzene aromatic ring, a heteroaromatic ring, or a non-benzene aromatic ring have been described above.

The first organic group and the second organic group are preferably bonded to each other directly or through a divalent linking group containing at least one bond selected from the group consisting of a carbon-carbon single bond, a carbon-carbon double bond, and a carbon-carbon triple bond.

More preferably, the first organic group is a benzene aromatic ring group, a heteroaromatic ring group, or a non-benzene aromatic ring group, the second organic group is a benzene aromatic ring group, a heteroaromatic ring group, or a non-benzene aromatic ring group, at least one carbon atom contained in an aromatic ring of the first organic group and at least one carbon atom contained in an aromatic ring of the second organic group are bonded to each other directly or through a divalent linking group containing at least one bond selected from the group consisting of a carbon-carbon single bond, a carbon-carbon double bond, and a carbon-carbon triple bond.

The first carbon atom is preferably bonded to the second carbon atom directly by a single bond or through a divalent linking group containing at least one bond selected from the group consisting of a carbon-carbon double bond and a carbon-carbon triple bond. The two aromatic rings form an extended π-electron system due to the above bonding forms. Such an onium salt can cause a reaction in which the π-electron system is shrinks by a photoreaction, and can improve a transmittance. Therefore, for example, when this onium salt is used as a photoacid generator for patterning of a thick film having a thickness of 10 μm or more, light can reach a deep portion of the film.

More preferably, at least one carbon atom contained in an aromatic ring of the first organic group and at least one carbon atom contained in an aromatic ring of the second organic group are bonded to each other directly or through an ethynylene group.

Specific preferable examples thereof include an onium salt represented by the above formula (a).

In the onium salt according to an embodiment, an absorption coefficient of the onium salt at a first wavelength is preferably reduced by that the onium salt absorps an electromagnetic wave or a particle beam having the first wavelength The electromagnetic wave or the particle beam is more preferably i-line, g-line, or h-line. Furthermore, the electromagnetic wave is preferably i-line. The first wavelength is preferably longer than 350 nm, and is preferably 365 nm.

In the cation portion of the onium salt according to one embodiment, the cation center such as a sulfur atom or an iodine atom has a relatively high electron accepting property. Therefore, electron transition to a lowest excited state or an excited state in the vicinity thereof comes to have a property of charge transfer from the it electron system bonded to the cation center to the cation center. By irradiating an absorption band having such a property of charge transfer with light, an electron moves to the cation center to improve an acid generation efficiency.

After the acid is generated, since the cation center is changed by a reaction, a probability of charge-transfer type electron transition is reduced, and an absorption coefficient at the first wavelength tends to be reduced. Therefore, by irradiation with an electromagnetic wave or a particle beam having the first wavelength, a light transmittance at the first wavelength is improved, the excitation light reaches a deep portion of a resist film, an acid generation efficiency of the entire film is improved, and time required for exposure can be shortened. Therefore, the onium salt according to one embodiment is suitable for patterning of a thick film.

In order to improve the probability of the charge-transfer type electron transition, at least one electron-donating group is preferably introduced into an aromatic ring (divalent organic group represented by W in the above formula (a)) linked to the cation center and/or an aromatic ring (monovalent organic group represented by Z in the above formula (a)) not linked to the cation center. Examples of the aromatic ring include a benzene aromatic ring, a heteroaromatic ring, and a non-benzene aromatic ring. As the aromatic ring linked to the cation center, any one of the benzene aromatic ring, the heteroaromatic ring, and the non-benzene aromatic ring may be introduced. Specific examples of the organic group containing an electron-donating group and an aromatic ring include the organic groups exemplified above.

As a method for improving the probability of the charge-transfer type electron transition, the degree of electron delocalization of a π electron system linked to the cation center is increased to raise the level of the highest occupied molecular orbital (HOMO). In the compounds of (17) to (22), a plurality of aromatic rings is bonded to each other directly or through a double bond or a triple bond. The plurality of aromatic rings is thereby interacted with each other to increase the degree of electron delocalization of the π electron system.

By further introducing at least one electron-donating group such as an alkoxy group into an aromatic ring (monovalent organic group represented by Z and/or divalent organic group represented by W in the above formula (a)) in the compounds of (17) to (22), contribution of the charge-transfer type transition in the electron transition is further increased to improve an acid generation efficiency.

Preferably, the onium salt according to one embodiment contains at least a first aromatic ring and a second aromatic ring which are the same as or different from each other, and one of carbon atoms forming a cyclic skeleton of the first aromatic ring is bonded directly to one carbon atom forming a cyclic skeleton of the second aromatic ring.

In this onium salt, the cation center of the cation portion is preferably bonded only to one of the first aromatic ring and the second aromatic ring. Furthermore, as in the compounds (17), (20), and (21), introduction of at least one electron-donating group into an aromatic ring of the first aromatic ring and the second aromatic ring, to which the cation center is not bonded, is preferable because charge-transfer type absorption having a transition moment in a long axis direction is enhanced, an electron can be supplied to the cation center, and the acid generation efficiency can be improved.

The onium salt according to an embodiment contains at least a first aromatic ring and a second aromatic ring which are the same as or different from each other, and one of carbon atoms forming a cyclic skeleton of the first aromatic ring is bonded to one carbon atom forming a cyclic skeleton of the second aromatic ring through at least one it electron system linking group such as a double bond or a triple bond.

In this onium salt, the cation center of the cation portion is preferably bonded only to one of the first aromatic ring and the second aromatic ring. Furthermore, as in the compounds (18), and (19), by introducing at least one electron-donating substituent into an aromatic ring of the first aromatic ring and the second aromatic ring, to which the cation center is not bonded, charge-transfer type absorption having a transition moment in a long axis direction is enhanced, an electron can be supplied to the cation center, and the acid generation efficiency can be improved.

<3> Method for Producing Onium Salt

A method for producing the onium salt represented by the above formula (a) is not particularly limited, but a method applying a known organic synthesis reaction can be used.

Among the onium salts represented by the above formula (a), examples of synthesis of a sulfonium salt in which A is a direct bond and Y is a sulfur atom include the following scheme.

In the following scheme, TfO— indicates trifluoromethanesulfonate.

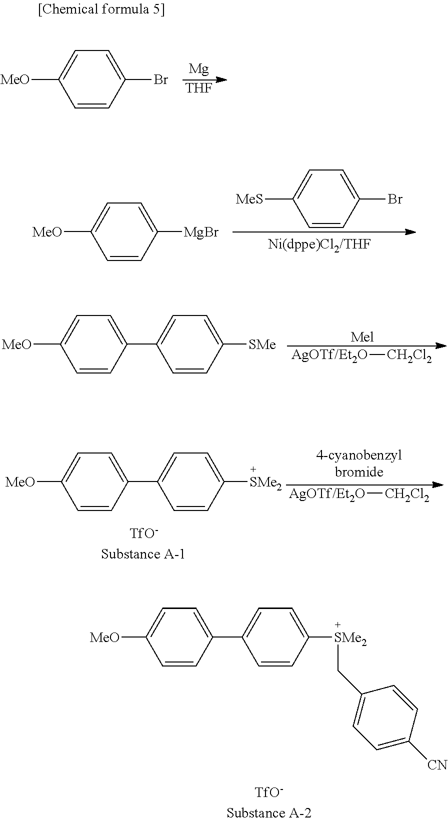

Hereinafter, specific synthetic methods will be described.

Magnesium is immersed in anhydrous tetrahydrofuran, and is activated with a small amount of dibromoethane. Thereafter, 5 ml of an anhydrous tetrahydrofuran solution containing 1.9 g of p-anisyl bromide is dropwise added slowly at room temperature. After stirring at room temperature for six hours, 0.3 g of [1,2-bis(diphenylphosphino) ethane] nickel(II) dichloride is added. Thereafter, 5 ml of an anhydrous tetrahydrofuran solution of 2.0 g of p-methylthiophenyl bromide is dropwise added slowly, and is heated and refluxed for 10 hours. After heating and refluxing, the temperature is returned to room temperature, and a solvent is distilled off from a solution obtained by allowing the mixture to pass through silica gel. The resulting solid is recrystallized with methanol and is dried to obtain 2.0 g of 4-methoxy-4′-methylthio-biphenyl. Subsequently, 1.15 g of 4-methoxy-4′-methylthio-biphenyl is dissolved in 10 ml of methylene chloride. Thereafter, 20 ml of a diethyl ether solution containing 1.5 g of silver triflate is added thereto for mixing. To this mixture, 10 ml of a methylene chloride solution of 0.7 g of iodomethane is dropwise added slowly and is stirred at room temperature for two hours. After stirring, 20 ml of acetonitrile is added to the mixture, and an insoluble matter is filtered. When a solvent of the filtrate becomes about ⅓, diisopropyl ether is added and the resulting mixture is stirred. At this time, a precipitate is generated. The precipitate is filtered, and then is dried to obtain 1.2 g of Substance A-1 as a target product.

0.3 g of magnesium is immersed in anhydrous tetrahydrofuran, and is activated with a small amount of dibromoethane. Thereafter, 5 ml of an anhydrous tetrahydrofuran solution containing 1.9 g of p-anisyl bromide is dropwise added slowly at room temperature. After stirring at room temperature for six hours, 0.3 g of [1,2-bis(diphenylphosphino) ethane] nickel(II) dichloride is added. Thereafter, 5 ml of an anhydrous tetrahydrofuran solution of 2.0 g of p-methylthiophenyl bromide is dropwise added slowly, and is heated and refluxed for 10 hours. After heating and refluxing, the temperature is returned to room temperature, and a solvent is distilled off from a solution obtained by allowing the mixture to pass through silica gel. The resulting solid is recrystallized with methanol and is dried to obtain 2.0 g of 4-methoxy-4′-methylthio-biphenyl. Subsequently, 1.15 g of 4-methoxy-4′-methylthio-biphenyl is dissolved in 10 ml of methylene chloride. Thereafter, 20 ml of a diethyl ether solution containing 1.5 g of silver triflate is added thereto for mixing. To this mixture, 10 ml of a methylene chloride solution of 1.0 g of p-cyanobenzyl bromide is added slowly and is stirred at room temperature for two hours. After stirring, 20 ml of acetonitrile is added to the mixture, and an insoluble matter is filtered. When a solvent of the filtrate becomes about ⅓, diisopropyl ether is added and the resulting mixture is stirred. At this time, a precipitate is generated. The precipitate is filtered, and then is dried to obtain 1.7 g of Substance A-2 as a target product.

Next, examples of synthesis of Substance A-3 will be described. In Substance A-3, all the substituents on a sulfur atom as the cation center are aromatic rings. Therefore, Substance A-3 exhibits excellent long-term stability.

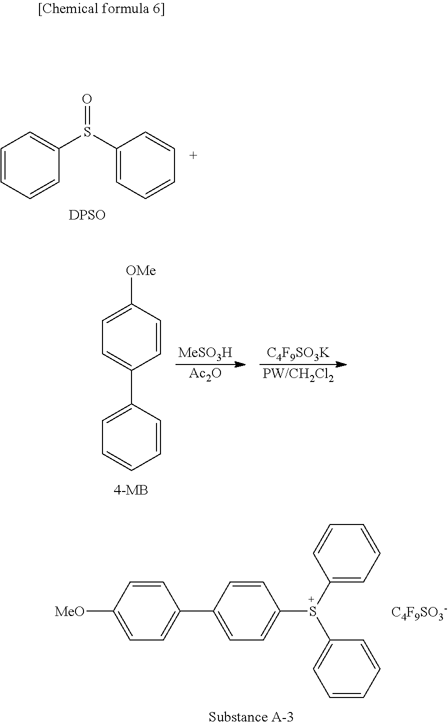

20 g of diphenyl sulfoxide (DPSO) and 22 g of 4-methoxy-biphenyl (4-MB) are dissolved in 162 g of acetic anhydride to prepare a solution of diphenyl sulfoxide and 4-methoxy-biphenyl. To this solution, 114 g of methanesulfonic acid is dropwise added so as not to raise the temperature. After dropwise addition, the resulting mixture is stirred for 16 hours at room temperature. Thereafter, 100 g of water is added slowly so as not to raise the temperature. Subsequently, 60 g of diisopropyl ether is added to the mixture containing water, the resulting mixture is extracted using a separatory funnel, the organic layer is discarded, and the aqueous layer is washed further using 60 g of diisopropyl ether. After the organic layer is discarded, 60 g of methylene chloride is added to the remaining aqueous layer, the resulting mixture is then subjected to separatory extraction, and the aqueous layer is discarded. To the remaining organic layer, 34 g of potassium perfluorobutanesulfonate is added, and the resulting mixture is stirred for two hours. 100 g of pure water is added. The resulting mixture is subjected to separatory extraction, and the aqueous layer is discarded. The organic layer is washed with 100 g of pure water until the pH thereof becomes neutral. After the resulting organic layer is concentrated, vacuum drying is performed to obtain Substance A-3 as 49 g of a solid.

Next, a synthetic scheme for a compound in which an aromatic ring to which the cation center is bonded and another aromatic ring are linked by a π-electron system linking group will be illustrated.

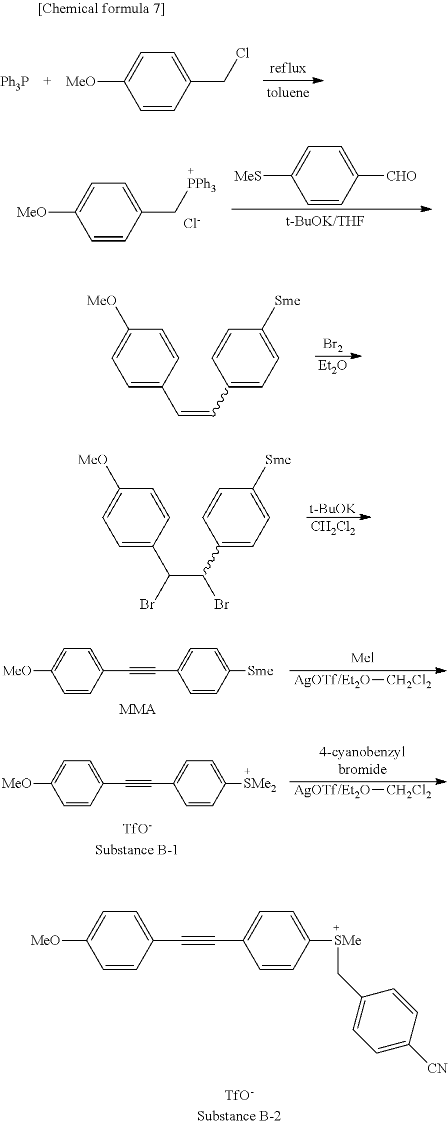

In the above scheme, TfO— indicates trifluoromethanesulfonate.

Hereinafter, synthetic schemes for Substances B-1 and B-2 will be described. MMA which is a diphenyl acetylene derivative is synthesized by a Wittig reaction, a bromination reaction, or a dehydrobromination reaction. Subsequently, 2.5 g of MMA is dissolved in 10 ml of methylene chloride. Thereafter, 20 ml of a diethyl ether solution containing 2.6 g of silver triflate is added thereto for mixing. To this mixture, 10 ml of a methylene chloride solution of 1.4 g of iodomethane is slowly added and is stirred at room temperature for two hours. After stirring, 20 ml of acetonitrile is added to the mixture, and an insoluble matter is filtered. When a solvent of the filtrate becomes about ⅓, diisopropyl ether is added and the resulting mixture is stirred. At this time, a precipitate is generated. The precipitate is filtered out and dried to obtain 1.2 g of substance B-1 as a target product.

Substance B-2 is synthesized as follows. 2.5 g of MMA is dissolved in 10 ml of methylene chloride. Thereafter, 20 ml of a diethyl ether solution containing 2.6 g of silver triflate is added thereto for mixing. To this mixture, 10 ml of a methylene chloride solution of 1.9 g of p-cyanobenzyl bromide is added slowly and is stirred at room temperature for two hours. After stirring, 20 ml of acetonitrile is added to the mixture, and an insoluble matter is filtered. When a solvent of the filtrate becomes about ⅓, diisopropyl ether is added and the resulting mixture is stirred. At this time, a precipitate is generated. The precipitate is filtered out and dried to obtain 3.7 g of substance B-2.

Chemical structures of the resulting sulfonium salt and iodonium salt can be identified by a general analysis method, for example, by liquid chromatography, 1H—NMR, 13C—NMR, IR, and/or elemental analysis.

<4> Photoacid Generator and Photosensitive Resin Composition Using the Same

An embodiment of the invention is a photoacid generator containing the onium salt. The photoacid generator of the invention releases an acid by irradiation with an electromagnetic wave or a particle beam having a first wavelength, and can cause decomposition or polymerization by acting on an acid-reactive organic substance. Therefore, the onium salt of the invention can be used preferably as a photoacid generator of a positive or negative photosensitive resin composition.

Specifically, the photoacid generator according to the embodiment of the invention can be used for a photosensitive resin composition containing a compound reacted by an acid.

Preferably, the compound reacted by an acid contains a protective group deprotected by an acid or performs crosslinking by an acid. That is, the compound reacted by an acid is preferably at least one selected from the group consisting of a compound containing a protective group deprotected by an acid and a crosslinking agent performing a crosslinking action by an acid.

The compound containing a protective group deprotected by an acid is a compound which changes solubility thereof with respect to a developing solution by deprotection of the protective group by an acid. For example, in a case of aqueous development using an alkali developing solution or the like, the compound containing a protective group deprotected by an acid is a compound which is insoluble in an alkali developing solution, but becomes soluble in the alkali developing solution by deprotection of a protective group in an exposed portion by an acid generated by the photoacid generator due to exposure.

In the invention, not only the alkali developing solution but also a neutral developing solution or an organic solvent developing solution may be used. Therefore, when an organic solvent developing solution is used, the compound containing a protective group deprotected by an acid is a compound which reduces solubility thereof with respect to the organic solvent developing solution by deprotection of a protective group in an exposed portion by an acid generated by the photoacid generator due to exposure.

Specific examples of the protective group deprotected by an acid include an ester group, an acetal group, a tetrahydropyranyl group, a siloxy group, and a benzyloxy group. As a compound containing the protective group, for example, a compound containing a styrene skeleton, methacrylate, or an acrylate skeleton having the protective group as a pendant group is preferably used.

The crosslinking agent performing a crosslinking action by an acid is a compound which changes solubility with respect to a developing solution by crosslinking by an acid. For example, in a case of aqueous development, the crosslinking agent performing a crosslinking action by an acid acts on a compound soluble in an aqueous developing solution to reduce solubility thereof with respect to the aqueous developing solution after crosslinking. Specific examples thereof include a crosslinking agent containing an epoxy group, an acetal group, an oxetanyl group, and the like. In this case, examples of a compound to be crosslinked include a compound containing a phenolic hydroxyl group.

A resist composition according to one aspect may further contain a polymer component having a weight average molecular weight of 2000 or more. As the polymer component, a component usually used in a resist composition is only required to be used. The content of the polymer component can be from 60 to 99% by mass in the total amount of the resist composition. As one of the polymer components, for example, hydroxystyrene is preferably contained. Hydroxystyrene has an easily-polarized phenol moiety, and therefore can stabilize a charge transfer excited state of the onium salt according to the aspect of the invention. For example, hydroxystyrene contributes to improvement of substrate adhesion, and can act as a compound to be crosslinked by a crosslinking agent performing a crosslinking action by an acid. Hydroxystyrene may be in an aspect of a polymer having hydroxystyrene as a constituent unit, may be in an aspect of polyhydroxystyrene, or may be in an aspect of a compound having a weight average molecular weight of less than 2000.

Hydroxystyrene is preferably contained in an amount of 40% by mass or more in the polymer components. This can improve a photoacid generation efficiency of the onium salt. The content of hydroxystyrene with respect to all the polymer components is more preferably 50% by mass or more, and still more preferably 60% by mass or more.

As the resist composition according to one aspect, more specifically, the following resist compositions can be exemplified.

Examples thereof include a resist composition containing the compound containing a protective group deprotected by an acid and the photoacid generator; and a resist composition containing a crosslinking agent performing a crosslinking action by an acid, a compound which changes solubility thereof with respect to a developing solution by reacting with the crosslinking agent, and a photoacid generator.

The content of the photoacid generator in the resist composition according to an aspect of the invention is preferably from 1 to 50 parts by mass, more preferably from 1 to 15 parts by mass, still more preferably from 5 to 8 parts by mass, and particularly preferably from 2 to 5 parts by mass with respect to 100 parts by mass of components of the resist composition excluding the photoacid generator. By the content of the photoacid generator within the above range in the resist composition, for example, it is possible to increase a transmittance of light even when the resist composition is used as a permanent film of an insulating film or the like of a display body or the like.

A photosensitive resin composition according to one aspect may contain another component within a range not impairing an effect of the invention in any aspect. Examples of a component which can be contained include the above polymer component and a known additive. For example, at least one selected from a known photoacid generator other than the above photoacid generator, a sensitizer, a quencher such as trioctylamine, a surfactant, a filler, a pigment, an antistatic agent, a flame retardant, a light stabilizer, an antioxidant, an ionic scavenger, a solvent, and the like may be added.

A method for preparing the photosensitive resin composition of the invention is not particularly limited, but the photosensitive resin composition can be prepared by a known method, for example, by mixing, dissolving, or kneading the above photoacid generator containing the onium salt, the above compound reacted by an acid, and any other component.

<5> Method for Producing Device

An embodiment of the invention is a method for producing a device, including:

a coating film forming step for forming a coating film on a substrate using the photosensitive resin composition containing the onium salt;

a photolithography step for exposing the coating film in a pattern shape using first light of an electromagnetic wave or a particle beam; and

a pattern forming step for developing the exposed coating film to obtain a photoresist pattern.

The first light used for exposure in the photolithography step is only required to be light which can generate an acid by activation of the onium salt of the invention, and means UV, a visible light beam, an X-ray, an electron beam, an ion ray, i-line, EUV, or the like. The photoacid generator according to the embodiment of the invention has a high sensitivity to i-line, and therefore the first light is preferably i-line.

An irradiation amount of light varies depending on the kind of each component and a blending ratio in a sensitive resin composition, a film thickness of a coating film, and the like, but is preferably 1 J/cm2 or less.

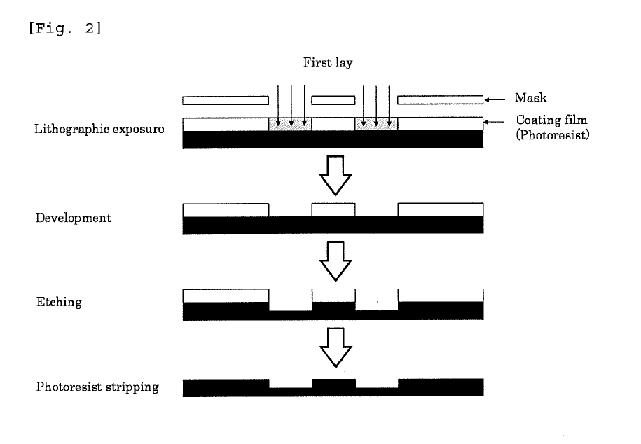

FIG. 2 illustrates a process for producing a device of an integrated circuit or the like using the photosensitive resin composition according to one aspect as a photoresist.

A silicon wafer is prepared. A surface of the silicon wafer is oxidized by heating the silicon wafer in the presence of oxygen gas. The photosensitive resin composition is applied on a Si wafer surface by spin coating to form a coating film. The coating film is pre-baked. After the Si wafer is pre-baked, the coating film and the silicon wafer are irradiated with first light having a wavelength of 220 nm or more through a mask. A typical light source for irradiation of a coating film as the first light is i-line or g-line.

Thereafter, a remaining film is removed. Irradiation time for the coating film can be shorter, and therefore deterioration of a device due to light irradiation is suppressed more than an existing photoresist.

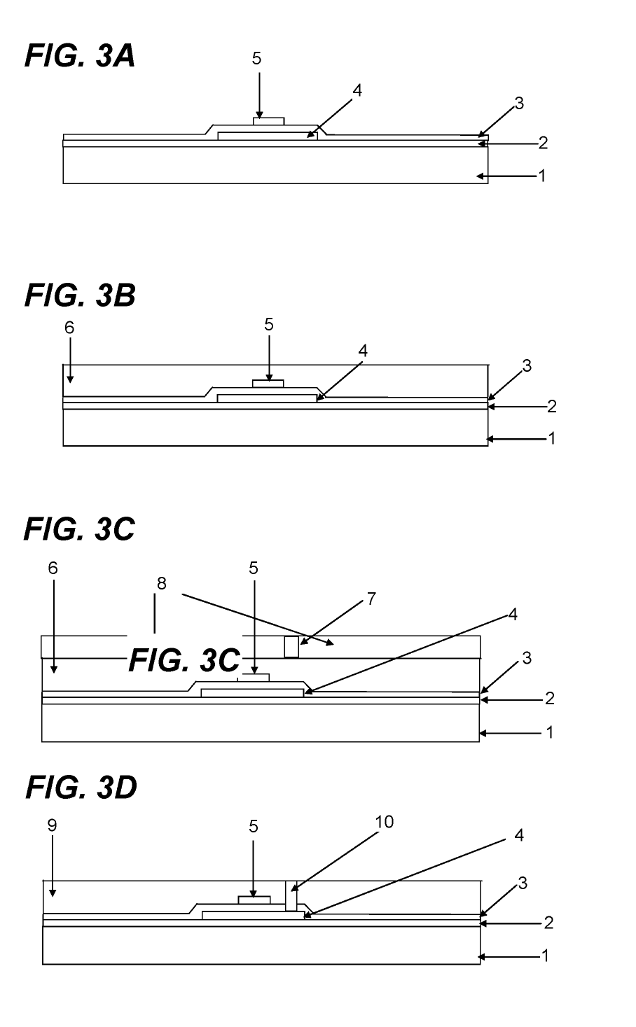

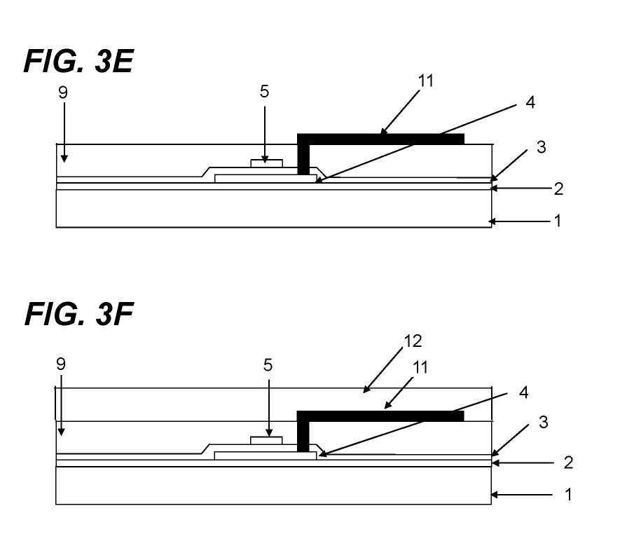

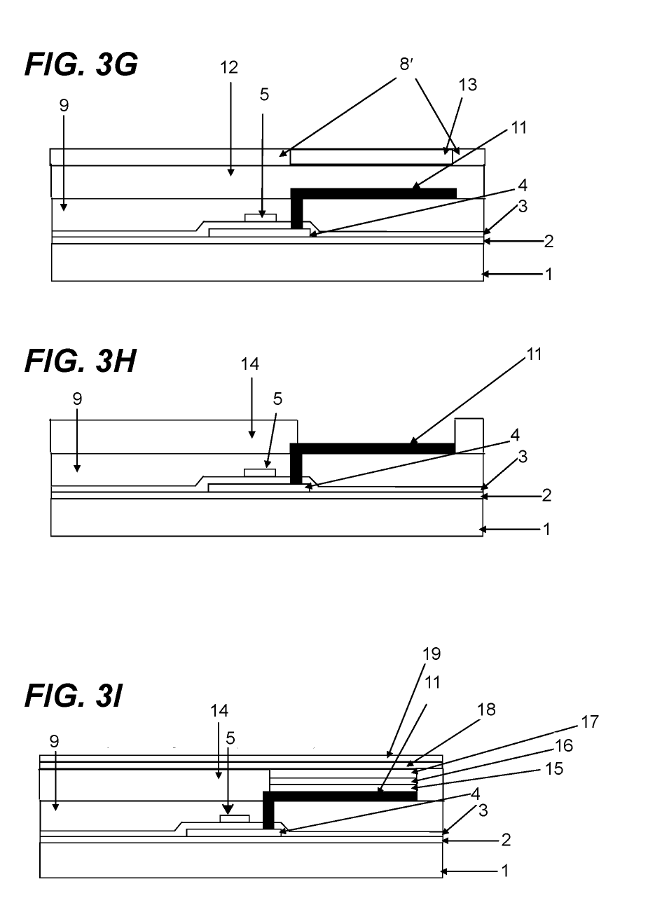

FIGS. 3A-3I illustrate a production process of an activated matrix type organic electroluminescent element.

In FIG. 3A, a lower layer 2 is formed on a substrate 1 such as a glass substrate, a quartz substrate, or a plastic substrate. A semiconductor film 4 formed by patterning is formed on the lower layer 2. Typically, the semiconductor film 4 is formed of low-temperature polysilicon. Amorphous silicon or a metal oxide can be used as a material of the semiconductor film 4. A gate insulating film 3 is formed so as to cover the semiconductor film 4. A gate electrode 5 is formed on the gate insulating film 3 such that the gate electrode 5 and the semiconductor film 4 face each other.

In FIG. 3B, the photosensitive resin composition according to an aspect of the invention is applied by spin coating to dispose a coating film 6 such that the coating film 6 covers the gate electrode 5 and the gate insulating film 3.

In FIG. 3C, the coating film 6 is pre-baked, and then is irradiated with light having a wavelength of 365 nm through a photomask 8. Only a part of the coating film 6 is exposed with light which has passed through an opening 7.

In FIG. 3D, a light-exposed portion of the coating film 6 is removed by development to form a contact hole 10. A heat treatment performed at a temperature higher than 150° C. converts the coating film 6 into a first interlayer insulating film, and then forms the coating film 6.

In FIG. 3E, a pixel electrode 11 electrically connected to the semiconductor film 4 is formed. Typically, the pixel electrode 11 is formed of indium tin oxide (ITO) or magnesium silver alloy.

In FIG. 3F, a coating film 12 is disposed by a spin coating process such that the coating film 12 covers the pixel electrode 11 and a first interlayer insulating film 9.

In FIG. 3G, the coating film 12 is pre-baked, and then is irradiated with light having a wavelength of 365 nm through a photomask 14. Only a part of the coating film 12 is exposed with light which has passed through an opening 13.

In FIG. 3H, a light-exposed portion of the coating film 12 is removed by development. A heat treatment performed at a temperature higher than 150° C. converts the coating film 12 into a second interlayer insulating film, and then forms the coating film 12.

In FIG. 3I, a hole transport layer 15, a light emitting layer 16, and a charge transport layer 17 are formed by a vacuum deposition method through a mask in this order. A common electrode 18 is formed on the charge transport layer 17 and a second interlayer insulating film 14. A protective film 19 is formed on the common electrode 18.

### EXAMPLES

Hereinbelow, the invention will be described more specifically based on Examples, but is not limited to the following Examples.

<Synthesis of Onium Salt>

### Example 1

(Synthesis of Onium Salt 1)

Onium salt 1 is synthesized as follows.

In the following scheme, TfO— indicates trifluoromethanesulfonate.

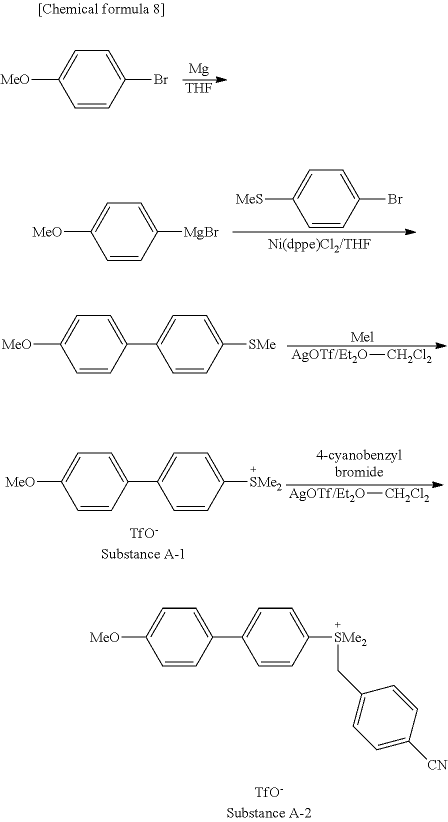

Hereinafter, specific synthetic methods will be described.

0.3 g of magnesium is immersed in anhydrous tetrahydrofuran, and is activated with a small amount of dibromoethane. Thereafter, 5 ml of an anhydrous tetrahydrofuran solution containing 1.9 g of p-anisyl bromide is dropwise added slowly at room temperature. After stirring at room temperature for six hours, 0.3 g of [1,2-bis(diphenylphosphino) ethane] nickel(II) dichloride is added. Thereafter, 5 ml of an anhydrous tetrahydrofuran solution of 2.0 g of p-methylthiophenyl bromide is dropwise added slowly, and is heated and refluxed for 10 hours. After heating and refluxing, the temperature is returned to room temperature, and a solvent is distilled off from a solution obtained by allowing the mixture to pass through silica gel. The resulting solid is recrystallized with methanol and is dried to obtain 2.0 g of 4-methoxy-4′-methylthio-biphenyl. Subsequently, 1.15 g of 4-methoxy-4′-methylthio-biphenyl is dissolved in 10 ml of methylene chloride. Thereafter, 20 ml of a diethyl ether solution containing 1.5 g of silver triflate is added thereto for mixing. To this mixture, 10 ml of a methylene chloride solution of 0.7 g of iodomethane is dropwise added slowly and is stirred at room temperature for two hours. After stirring, 20 ml of acetonitrile is added to the mixture, and an insoluble matter is filtered. When a solvent of the filtrate becomes about ⅓, diisopropyl ether is added and the resulting mixture is stirred. At this time, a precipitate is generated. The precipitate is filtered, and then is dried to obtain 1.2 g of onium salt 1 (Substance A-1) as a target product.

### Example 2

(Synthesis of Onium Salt 1)

Onium salt 1 is synthesized as follows.

0.3 g of magnesium is immersed in anhydrous tetrahydrofuran, and is activated with a small amount of dibromoethane. Thereafter, 5 ml of an anhydrous tetrahydrofuran solution containing 1.9 g of p-anisyl bromide is dropwise added slowly at room temperature. After stirring at room temperature for six hours, 0.3 g of [1,2-bis(diphenylphosphino) ethane] nickel(II) dichloride is added. Thereafter, 5 ml of an anhydrous tetrahydrofuran solution of 2.0 g of p-methylthiophenyl bromide is dropwise added slowly, and is heated and refluxed for 10 hours. After heating and refluxing, the temperature is returned to room temperature, and a solvent is distilled off from a solution obtained by allowing the mixture to pass through silica gel. The resulting solid is recrystallized with methanol and is dried to obtain 2.0 g of 4-methoxy-4′-methylthio-biphenyl. Subsequently, 1.15 g of 4-methoxy-4′-methylthio-biphenyl is dissolved in 10 ml of methylene chloride. Thereafter, 20 ml of a diethyl ether solution containing 1.5 g of silver triflate is added thereto for mixing. To this mixture, 10 ml of a methylene chloride solution of 1.0 g of p-cyanobenzyl bromide is added slowly and is stirred at room temperature for two hours. After stirring, 20 ml of acetonitrile is added to the mixture, and an insoluble matter is filtered. When a solvent of the filtrate becomes about ⅓, diisopropyl ether is added and the resulting mixture is stirred. At this time, a precipitate is generated. The precipitate is filtered, and then is dried to obtain 1.7 g of onium salt 2 (Substance A-2) as a target product.

### Example 3

(Synthesis of Onium Salt 3)

Onium salt 3 is synthesized as follows.

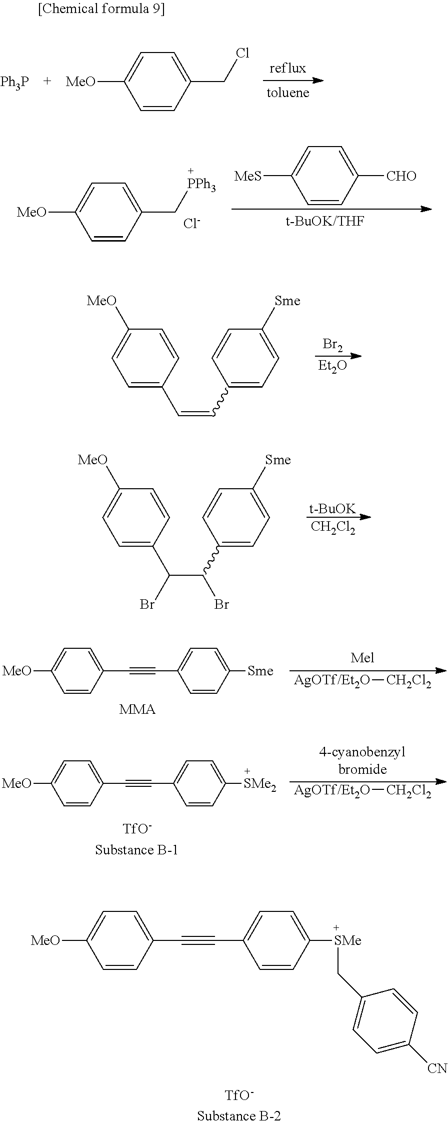

In the above scheme, TfO— indicates trifluoromethane sulfonate.

MMA which is a diphenyl acetylene derivative is synthesized by a Wittig reaction, a bromination reaction, or a dehydrobromination reaction. Subsequently, 2.5 g of MMA is dissolved in 10 ml of methylene chloride. Thereafter, 20 ml of a diethyl ether solution containing 2.6 g of silver triflate is added thereto for mixing. To this mixture, 10 ml of a methylene chloride solution of 1.4 g of iodomethane is slowly added and is stirred at room temperature for two hours. After stirring, 20 ml of acetonitrile is added to the mixture, and an insoluble matter is filtered. When a solvent of the filtrate becomes about ⅓, diisopropyl ether is added and the resulting mixture is stirred. At this time, a precipitate is generated. The precipitate is filtered out and dried to obtain 1.2 g of onium salt 3 (Substance B-1) as a target product.

### Example 4

(Synthesis of Onium Salt 4)

Onium salt 4 is synthesized as follows.

2.5 g of MMA obtained in Example 3 is dissolved in 10 ml of methylene chloride. Thereafter, 20 ml of a diethyl ether solution containing 2.6 g of silver triflate is added thereto for mixing. To this mixture, 10 ml of a methylene chloride solution of 1.9 g of p-cyanobenzyl bromide is added slowly and is stirred at room temperature for two hours. After stirring, 20 ml of acetonitrile is added to the mixture, and an insoluble matter is filtered. When a solvent of the filtrate becomes about ⅓, diisopropyl ether is added and the resulting mixture is stirred. At this time, a precipitate is generated. The precipitate is filtered out and dried to obtain 3.7 g of onium salt 4 (Substance B-2).

### Example 5

(Synthesis of Onium Salt 5)

Onium salt 5 is synthesized as follows.

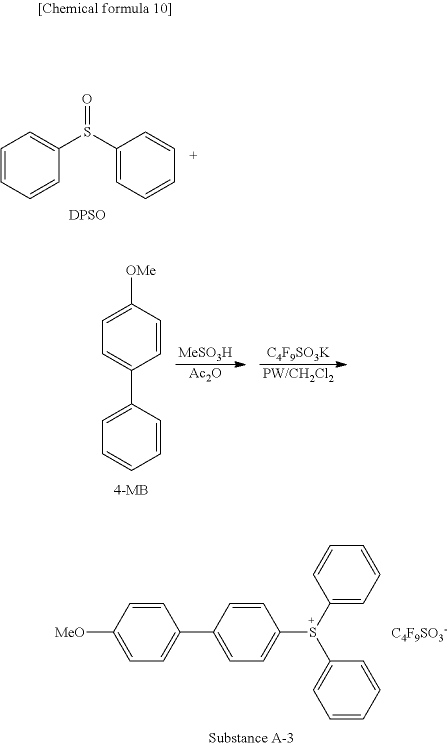

20 g of diphenyl sulfoxide (DPSO) and 22 g of 4-methoxy biphenyl (4-MB) are dissolved in 162 g of acetic anhydride to prepare a solution of diphenyl sulfoxide and 4-methoxy biphenyl. To this solution, 114 g of methanesulfonic acid is dropwise added so as not to raise the temperature. After dropwise addition, the resulting mixture is stirred at room temperature for 16 hours. Thereafter, 100 g of water is dropwise added slowly so as not to raise the temperature. Subsequently, 60 g of diisopropyl ether is added to the mixture containing water, the resulting mixture is extracted using a separatory funnel, the organic layer is discarded, and the aqueous layer is washed further using 60 g of diisopropyl ether. After the organic layer is discarded, 60 g of methylene chloride is added to the remaining aqueous layer, the resulting mixture is then subjected to separatory extraction, and the aqueous layer is discarded. To the remaining organic layer, 34 g of potassium perfluorobutane sulfonate is added, and the resulting mixture is stirred for two hours. 100 g of pure water is added. The resulting mixture is subjected to separatory extraction, and the aqueous layer is discarded. The organic layer is washed with 100 g of pure water until the pH thereof becomes neutral. After the resulting organic layer is concentrated, vacuum drying is performed to obtain onium salt 5 (Substance A-3) as 49 g of a solid.

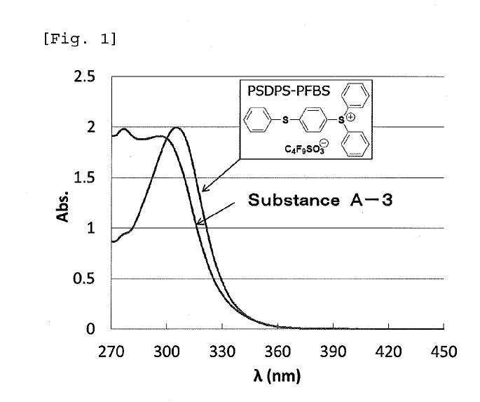

FIG. 1 illustrates an absorption spectrum of the resulting onium salt 5 (Substance A-3). An absorption spectrum of PSDS-PFBS widely used as an i-line photoacid generator is also illustrated for comparison. An absorption spectrum of Substance A-3 reaches the vicinity of 365 nm like PSDS-PFB S.

<Preparation of Photoresist>

A photoresist sample is prepared using 9.0 mg of any one of onium salts 1 to 5 obtained in Examples 1 to 5, 225 mg of polymer A below, and 225 mg of polymer B below. A sensitivity is evaluated as follows by using this photoresist sample as an evaluation sample. Comparative Example 1 is an example in which PSDS-PFBS widely used as an i-line photoacid generator is used in place of onium salts 1 to 5.

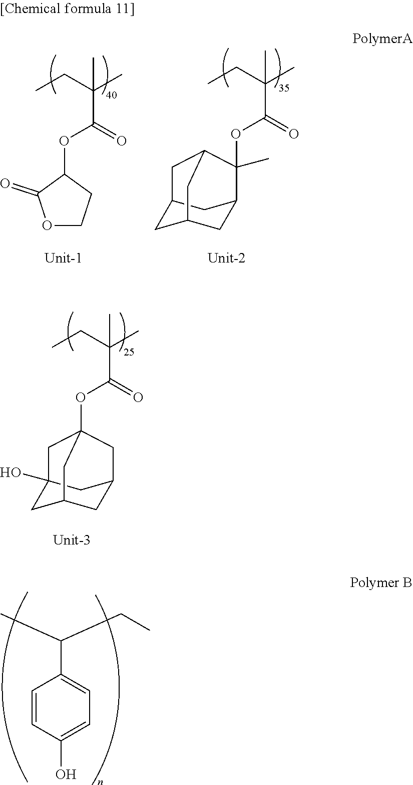

<Sensitivity Evaluation>

Before the evaluation sample is applied on a Si wafer, hexamethyldisilazane (HMDS, Tokyo Chemical Industry Co., Ltd.) is spin-coated on a surface of the Si wafer at 2000 rpm for 20 seconds, and is baked at 110° C. for one minute. Thereafter, the evaluation sample is spin-coated on the surface of the Si wafer treated with the HMDS at 2000 rpm for 20 seconds to form a coating film. The coating film is pre-baked at 110° C. for 60 seconds. Thereafter, the coating film of the evaluation sample is exposed with i-line (365 nm) output from a UV light emitting device (HMW-661C-3 manufactured by Oak Manufacturing Co., Ltd.). After light exposure with i-line, post-baking (PEB) is performed at 110° C. for 60 seconds. The coating film is developed by NMD-3 (2.38% tetra-methyl ammonium hydroxide manufactured by Tokyo Ohka Kogyo Co., Ltd.) at 25° C. for 20 seconds, and is washed with deionized water for 10 seconds. The thickness of the coating film measured with a film thickness measurement tool is approximately 500 nm.

A sensitivity (E0 sensitivity) is evaluated by measuring a dose size for forming a coating film with a pattern formed of a 100 μm line having a coating film thickness of not zero and a 100 μm space having a coating film thickness of zero using UV exposure. The dose of the E0 sensitivity is calculated by illuminance measurement of a UV source with a 365 nm illuminometer. Table 1 illustrates results thereof.

Onium salt 5 of Example 5 has a high transmittance almost equal to PSDS-PFBS in Comparative Example 1 as a typical high transmittance photoacid generator, but exhibits a sensitivity as high as 1.3 times or more that of PSDPS-PFBS.

<Evaluation of Molar Absorption Coefficient>

A molar absorption coefficient at 365 nm before irradiation with i-line is evaluated for each of onium salts 1 to 5 and PSDS-PFBS. Table 1 illustrates results thereof. The molar absorption coefficient is indicative of a transmittance.

TABLE 1

| | | | Molar adsorption coefficient |
|---|---|---|---|
| | | | (before irradiation with |
| | PAG | E₀mJ/cm² | i-line) |
| Example 1 | Onium salt 1 | >600 | 0.9E+05 |
| Example 2 | Onium salt 2 | 400 | 1.30E+05 |
| Example 3 | Onium salt 3 | <50 | 2.50E+05 |
| Example 4 | Onium salt 4 | <30 | 3.30E+05 |
| Example 5 | Onium salt 5 | 285 | 1.52E+05 |
| Comparative | PSDS-PFBS | 345 | 1.28E0+05 |
| Example 1 | | | |

Onium salts 1, 2, and 5 have proper molar absorption coefficients, and therefore are considered to have very high transmittances. An onium salt in which two aromatic rings of onium salts 3 and 4 or the like are linked by a π-electron system linking group such as a triple bond, and the cation center of the onium is bonded to one aromatic ring of the two aromatic rings exhibits a very high sensitivity. In onium salt 5, the molar absorption coefficient after irradiation with i-line is about ⅕ of that before irradiation therewith. Also in onium salts 1 to 4, attenuation of the absorption coefficient is observed at a wavelength longer than 350 nm as in onium salt 5.

### INDUSTRIAL APPLICABILITY

An onium salt according to one embodiment has a high sensitivity to i-line, and has a high transmittance of light. Therefore, the onium salt can be also used for patterning of a thick film by using a photosensitive resin composition containing the onium salt as a photoacid generator for a photoresist.

### REFERENCE SIGNS LIST

1 substrate

2 lower layer

3 insulating film

4 semiconductor film

5 gate electrode

6 coating film

7 opening

8, 8′ photomask

9 first interlayer insulating film

10 contact hole

11 pixel electrode

12 coating film

13 opening

14 second interlayer insulating film

15 hole transport layer

16 light emitting layer

17 charge transport layer

18 common electrode

19 protective film

1. The onium salt of claim 11, wherein:
a chemical structure which is represented by the following formula (a),
Z-A-W—Y⁺(R)ₙX⁻ (a)
where:
Z is a first monovalent organic group having a cyclic structure including a conjugated π-electron system optionally including at least one substituent;
W is a divalent organic group having a cyclic structure including a conjugated π-electron system optionally including at least one substituent;
A is the direct bond or the divalent linking group including at least one bond selected from the group consisting of the carbon-carbon single bond, the carbon-carbon double bond and the carbon-carbon triple bond;
any substituent of Z and W may form a cyclic structure saturated or partially saturated with A;
the cyclic structure includes at least one atom included in each of Z and W;
Y is an iodine atom or a sulfur atom;
when Y is an iodine atom, n=1;
when Y is a sulfur atom, n=2;
R may be the same as or different from one another;
each of R is a second monovalent organic group having one or more carbon atoms and optionally containing a substituent;
when A is the carbon-carbon double bond, at least one R is the third aromatic ring optionally containing at least one substituent, and
X⁻ is a monovalent anion.

2. The onium salt according to claim 1, wherein:
when A is the carbon-carbon single bond, the carbon-carbon double bond, or the carbon-carbon triple bond, Z is any organic group selected from the group consisting of the monovalent benzene aromatic ring group, the monovalent heteroaromatic ring group and the monovalent non-benzene aromatic ring group.

3. The onium salt according to claim 1, wherein:
W is any organic group selected from the group consisting of a divalent benzene aromatic ring group, a divalent heteroaromatic ring group and a divalent non-benzene aromatic ring group.

4. The onium salt according to claim 1, wherein at least one of Z and W includes at least one electron-donating group.

5. The onium salt according to claim 1, wherein Z is a phenyl group and/or W is a phenylene group.

6. The onium salt according to claim 4, wherein the at least one electron-donating group is any one selected from the group consisting of an alkyl group (—R¹), an aryl group (—Ar¹), an alkoxy group (—OR¹), an aryloxy group (—OAr¹), a hydroxy group, a thiol group, an alkylthio group (—SR¹), an amino group, an alkylamino group (—NHR¹), a dialkylamino group (—NR¹R²), an arylamino group (—NHAr¹), a diarylamino group (—NAr¹Ar²) and an N-alkyl-N-arylamino group (—NR¹Ar¹).

7. The onium salt according to claim 4, wherein the at least one electron-donating group is bonded directly to the conjugated π-electron system of Z.

8. The onium salt according to claim 1, wherein:
A is a direct bonds or
A is an ethynylene group.

9. The onium salt according to claim 1, wherein the monovalent organic group in R is monovalent hydrocarbon group or an aryl group.

10. The onium salt according to claim 1, wherein X⁻ is an anion selected from the group consisting of CF₃CO₂⁻, CH₃CO₂⁻, CF₃CF₂C₄H₄SO₃, CH₃SO₃, (C₆F₅)₄B⁻, SbF₆⁻, PF₆⁻, BF₄⁻, CF₃SO₃, HSO₄, (CF₃CF₂)₃PF₃⁻, (CF₃CF₂)₂PF₄⁻, (CF₃CF₂)PF₅⁻, ((CF₃)₂C₆H₃)₄B⁻, (C₆F₅)₄Ga⁻, ((CF₃)₂C₆H₃)₄Ga⁻, a nonafluorobutanesulfonic acid anion, a butanesulfonic acid anion, a camphorsulfonic acid anion, a benzenesulfonic acid anion, a p-toluenesulfonic acid anion, (CF₃SO₂)₃C⁻, (CF₃SO₂)₂N⁻ and (C₄F₉SO₂)₂N⁻.

11. An onium salt comprising:
a cation portion and an anion portion,
wherein:
the cation portion includes at least a first aromatic ring, a second aromatic ring and a cation center;
the cation center is bonded to the first aromatic ring ;
a first carbon atom included in the first aromatic ring is bonded to a second carbon atom included in the second aromatic ring by a direct bond between the first carbon atom and the second carbon atom or through a divalent linking group including at least one bond selected from the group consisting of a carbon-carbon single bond, a carbon-carbon double bond and a carbon-carbon triple bond;
when the first carbon atom is bonded to the second carbon atom by the direct bond between the first carbon atom and the second carbon atom, the second aromatic ring is selected from the group consisting of a monovalent benzene aromatic ring group, a monovalent heteroaromatic ring group, and a monovalent non-benzene aromatic ring group that optionally includes at least one substituent;
the monovalent benzene aromatic ring group is selected from the group consisting of a phenyl group, a biphenyl group, a terphenyl group, a quaterphenyl group, a naphthyl group, a phenanthrenyl group, a pentalenyl group, an indenyl group, an indacenyl group, an acenaphthyl group, a fluorenyl group, a heptalenyl group, a pyrenyl group, and a chrysenyl group; and
when the first carbon atom is bonded to the second carbon atom through the divalent linking group is the carbon-carbon double bond, the cation center is further bonded to at least a third aromatic ring.

12. The onium salt according to claim 11, wherein an absorption coefficient of the onium salt at a first wavelength is reduced by an irradiation of the onium salt with an electromagnetic wave having the first wavelength or a particle beam.

13. through 15. (canceled)

16. The onium salt according to claim 11, wherein at least one of the first aromatic ring and the second aromatic ring is a benzene ring.

17. The onium salt according to claim 11, wherein:
the cation portion further contains an electron-donating group; and
the electron-donating group is bonded to at least one of the first aromatic ring and the second aromatic ring.

18. The onium salt according to claim 17, wherein the electron-donating group is bonded to an aromatic ring of the first aromatic ring and the second aromatic ring that is not bonded to the cation center.

19. A photoacid generator comprising the onium salt according to claim 11.

20. A photosensitive resin composition comprising:
the photoacid generator according to claim 19; and
a compound that is to react with an acid.

21. The photosensitive resin composition according to claim 20, wherein:
the compound is to be polymerized by an acid
the compound includes a protective group to be deprotected by an acid; or
the compound is to be cross-linked by an acid.

22. A method for producing a device, the method comprising:
forming a coating film on a substrate utilizing the photosensitive resin composition according to claim 20;
exposing the coating film in a pattern shape utilizing first light of an electromagnetic wave or a particle beam; and
developing the coating film to obtain a photoresist pattern after exposing the coating film.

23. (canceled)

24. The onium salt of claim 1, wherein:
the chemical structure includes the cation portion and the anion portion;
the cation portion includes Z, W, A, and Y⁺;
the anion portion includes X⁻;
the cation portion includes the first aromatic ring, the second aromatic ring and the cation center;
the first aromatic ring is W; and
the second aromatic ring is Z;
when A is the direct bond and the direct bond is a bond between the first carbon atom included in W and the second carbon atom included in Z, Z is selected from the group consisting of the monovalent benzene aromatic ring group, the monovalent heteroaromatic ring group, and the monovalent non-benzene aromatic ring group that optionally includes at least one substituent.
